(19) United States
(12) Patent Application Publication
(10) Pub. No.: US 2004/0013634 A1
(43) Pub. Date: Jan. 22, 2004

(54) Shaving composition containing polyglyceryl ester surfactant

(75) Inventors: Alexander Novikov, Framingham, MA (US); Stephen H. Thong, Needham, MA (US)

Correspondence Address: PATENT AND TRADEMARK COUNSEL, THE GILLETTE COMPANY, 800 BOYLSTON STREET, BOSTON, MA, 02199, US

(73) Assignee: The Gillette Company

(21) Appl. No.: 10/199,408

(22) Filed: Jul. 19, 2002

(51) Int. Cl. A61K 7/15

(52) U.S. Cl. 424/73

(57) ABSTRACT

Disclosed are shaving compositions that contain a polyglyceryl fatty ester surfactant and a fatty alcohol. In particular the shaving composition comprises, in percent by weight, about 65% to about 94% water, about 1% to about 10% of a C₁₂₋₂₂ fatty alcohol, about 1% to about 6% of a volatile hydrocarbon propellant, and about 1% to about 10% of a polyglyceryl fatty ester surfactant. Preferably the shaving composition is substantially free of anionic surface active agents such as soap or detergent.

### BACKGROUND OF THE INVENTION

The present invention relates to shaving compositions that contain a polyglyceryl fatty ester surfactant and a fatty alcohol.

Currently, a widely used form of shaving preparation is the type referred to as aerosol shave cream or foam. These instant foams are dispensed from pressurized aerosol containers in the form of a rich lather for spreading by hand on the area to be shaved. An instant foam typically comprises water, a dispersed or solubilized surface-active agent (e.g., an anionic, cationic, amphoteric, or nonionic surface-active agent, or combination thereof), and a propellant/foaming agent. They may also include ingredients such as foam builders, foam stabilizers, emollients, viscosity modifiers, lubricants, humectants, preservatives, and fragrances.

Generally, the instant foams include anionic surface-active agents such as soaps (erg., sodium stearate) or interrupted soaps (e.g., sodium stearoyl sarcosinate) in order to achieve an adequate level of lathering which is desired by the typical user. In addition, they often also include anionic detergents. These anionic surface-active agents, particularly the soaps, do not provide adequate skin lubrication to achieve a comfortable shave and tend to remove the lipid protective barrier on the skin causing the skin to feel dry and unprotected.

U.S. Pat. No. 4,046,874 (Gabby) discloses a soap-free aerosol shaving cream containing a polyglycerol fatty ester (e.g., triglycerol monostearate, decaglycerol distearate) and a water insoluble pulverulent bodying agent such as dextran, cellulose, talc, silica, sodium silicoaluminate and clay. However, such a product would not have good application aesthetics.

It would be highly desirable to provide an aerosol shaving composition that provides good foaming characteristics and excellent lubricity without drying or irritating the skin. It is especially desirable to provide such a composition that is substantially free of soap and/or other anionic surfactant.

### SUMMARY OF THE INVENTION

The present invention embraces shaving compositions that contain a polyglyceryl fatty ester surfactant and a fatty alcohol. In particular the shaving composition comprises, in percent by weight, about 65% to about 94% water, about 1% to about 10% of a C₁₂₋₂₂ fatty alcohol, about 1% to about 6% of a volatile hydrocarbon propellant, and about 1% to about 10% of a polyglyceryl fatty ester surfactant. Preferably the shaving composition is substantially free of anionic surface active agents such as soap or detergent. In addition, the shaving composition preferably is substantially free of water insoluble pulverulent bodying agents such as dextran, cellulose, talc, silica, sodium silicoaluminate and clay. It is also desirable that the shaving composition is substantially free of polyethoxylated fatty alcohols.

### DETAILED DESCRIPTION OF THE INVENTION

The present invention embraces shaving compositions that contain a polyglyceryl fatty ester surfactant and a fatty alcohol. The term “fatty”, as used herein, means a hydrocarbon chain having 12-22 carbon atoms (C₁₂₋₂₂). The chain may be straight or branched and may be saturated or unsaturated (typically one or two double bonds in the chain). The fatty alcohol may be any C₁₂₋₂₂ fatty alcohol. It provides body, emolliency and a rich creamy skin feel. Typical fatty alcohols include lauryl, myristyl, cetyl, coconut, cetearyl, stearyl, oleyl, arachidyl, and behenyl alcohol. The fatty alcohol should be present in the shaving composition in an amount of about 1% to about 10% by weight, preferably about 2% to about 6% by weight.

The polyglyceryl fatty ester surfactant provides a good lather or foam and a soft, moisturizing skin feel. Ideally, it will have an HLB of at least 9 or greater, preferably at least 10 or greater, more preferably at least 12 or greater. It should be present in the shaving composition in an amount of about 1% to about 10% by weight, preferably about 2% to about 6% by weight.

The polyglyceryl fatty ester surfactant has the formula

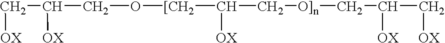

wherein n is 1 to 10, preferably 4 to 8, and

X is a hydrogen atom or a long chain acyl group (as defined below), provided that at least one X is a long chain acyl group, and preferably no more than three X's are long chain acyl groups. Most preferably no more than two X's are long chain acyl groups. The long chain acyl group may be derived from a C₁₂-₂₂ fatty acid or an N-fatty acyl-neutral amino acid. (By the terminology the acyl group may be derived from a particular acid is meant simply that the OH group of the acid R—COOH is removed to yield the corresponding acyl group R—CO—.) Thus, the polyglyceryl fatty ester surfactant may be a polyglyceryl C₁₂₋₂₂ fatty acid ester or a polyglyceryl N-fatty acyl aminoacid ester.

The long chain acyl group is preferably derived from a C₁₂₋₂₂ fatty acid. Thus, typical long chain acyl groups of this type include, for example, lauroyl, myristoyl, palmitoyl, stearoyl, isostearoyl, oleoyl, linoleoyl, and behenoyl. As mentioned above, the polyglyceryl ester will have at least one fatty acyl group, but preferably no more than three fatty acyl groups, in the molecule. Accordingly, when the acyl group is oleoyl, then the polyglyceryl ester will be named polyglyceryl-(n+2) (mono-, di- or tri-) oleate, where (n+2) is the degree of polymerization of the glycerol moiety (where n is as defined above) and is, thus, an integer from 3 to 12, preferably 6 to 10. Examples of suitable polyglyceryl fatty esters include, but are not limited to, polyglyceryl-10 oleate (also known as decaglycerol monooleate), polyglyceryl-6 stearate (also known as hexaglycerol monostearate), polyglyceryl-10 stearate (also known as decaglycerol monostearate), polyglyceryl-8 dipalmitate (also known as octaglycerol dipalmitate), polyglyceryl-10 dipalmitate (also known as decaglycerol dipalmitate), polyglyceryl-10 behenate (also known as decaglycerol monobehenate), and polyglyceryl-12 trilaurate (also known as dodecaglycerol trilaurate).

Alternatively, the long chain acyl group may also be derived from an N-fatty acyl-neutral amino acid. The fatty acyl moiety may be a C₁₂₋₂₂ fatty acyl group as described above (e.g., lauroyl, myristoyl, stearoyl, oleoyl, etc.). The neutral amino acid may be any short chain (i.e., 2 to 4 carbon atoms) amino acid such as glycine, alanine, β-alanine, aminobutyric acid, a-aminobutyric acid, N-methyl-β-alanine, and sarcosine. Sarcosine is preferred, in which case the long chain acyl group is N-fatty acyl-sarcosyl, and the polyglyceryl ester is a polyglyceryl N-fatty acyl-sarcosinate. Thus, suitable long chain acyl groups of this type include, for example, those derived from N-lauroyl-sarcosine, N-myristoyl-sarcosine, N-stearoyl-sarcosine, N-oleoyl-sarcosine, etc. Accordingly, examples of suitable polyglyceryl N-fatty acyl amino acid esters include, but are not limited to, polyglyceryl-10 N-stearoyl-sarcosinate (also known as decaglycerol mono-(N-stearoyl-sarcosinate)), polyglyceryl-10 N-lauroyl-sarcosinate (also known as decaglycerol mono-(N-lauroyl-sarcosinate)), polyglyceryl-8 di-(N-myristoyl-sarcosinate) (also known as octaglycerol di-(N-myristoyl-sarcosinate)), and polyglyceryl-12 tri-(N-lauroyl-sarcosinate) (also known as dodecaglycerol tri-(N-lauroyl-sarcosinate)).

The main component of the shaving composition is water, which typically makes up about 65% to about 94%, preferably about 75% to about 90% of the composition by weight.

A volatile hydrocarbon propellant is included in the composition to perform two functions: (1) to propel the shaving composition from its container and (2) to cause the shaving preparation to foam. Suitable volatile hydrocarbon propellants will have a vapor pressure of 15 to 80 psig, preferably 30 to 70 psig, at 20° C. Such propellants typically include a volatile hydrocarbon or a mixture of volatile hydrocarbons (typically with 3 to 5 carbon atoms) and/or a halohydrocarbon (typically with 1 to 3 carbon atoms). The term volatile hydrocarbon is intended to include the halohydrocarbons. A preferred propellant has the industry designation propellant A-46 and is a mixture of n-butane, isobutane and propane with a vapor pressure of 46 psig at 20° C. Other suitable propellants include propellant A-31 (isobutane), propellant 152A (1,1-difluoroethane) and propellant A-70 (a mixture of propane and isobutane). The propellant typically comprises about 1% to 6%, preferably about 2% to 5%, by weight of the composition.

An advantageous shaving composition of the present invention is one which contains a minimum of ingredients. This provides the benefits of simple formulation and reduced costs. A simple formulation consists essentially of the aforementioned four components, namely water, fatty alcohol, propellant and polyglyceryl fatty ester surfactant. The term “consist(ing) essentially of” is not intended to exclude materials that do not affect the basic and fundamental characteristics of the composition. For example, one might normally include in a simple formula a fragrance, a colorant, a preservative (e.g., antimicrobial or antioxidant), or even a small amount (e.g., about 2% or less) of an agent to improve application aesthetics, but none of these are viewed as an ingredient that materially affects the basic and fundamental characteristics of the composition. Preferably the total amount of all such non-essential ingredients will be less than 5%, more preferably less than about 2.5%, of the composition by weight.

Although not necessary to forming a useful shaving composition, other cosmetic ingredients may be advantageously added to improve the application aesthetics and/or achieve other shave benefits. For example, the composition may include one or more of the following components: beard wetting agents, skin conditioning agents (e.g., vitamins A, C and E, aloe, allantoin, panthenol, alpha-hydroxy acids, phospholipids, triglycerides, botanical oils, amino acids), foam building agents, emollients, humectants (e.g., glycerin, sorbitol, propylene glycol), fragrances, colorants, antioxidants, preservatives, etc. The shaving composition may also include a minor amount of a non-ionic and/or amphoteric surfactant.

In particular, it may be advantageous to include a sorbitan fatty ester or a sucrose fatty ester or a propoxylated fatty amide. Each of these materials may be included in the shaving composition in an amount of about 0.5% to about 5%, preferably about 1% to about 4%, by weight. Sorbitan fatty esters include sorbitan stearate, sorbitan oleate, sorbitan isostearate, sorbitan laurate, sorbitan dioleate, etc. Sucrose fatty esters include sucrose stearate, sucrose oleate, sucrose isostearate, sucrose cocoate, sucrose distearate, etc. The sorbitan esters and sucrose esters may be mixtures of mono-, di- and tri-esters. The propoxylated fatty amide will typically have from 1 to 3 propoxyl groups attached to a hydroxyloweralkyl fatty amide. Thus, suitable propoxylated fatty amides include, for example, PPG-2-hydroxyethyl coco/isostearamide, PPG-3-hydroxyethyl linoleamide, and PPG-2-hydroxyethyl cocamide.

Preferably the shaving composition is substantially free of anionic surface active agents such as soap or detergent. In addition, the shaving composition preferably is substantially free of water insoluble pulverulent bodying agents such as dextran, cellulose, talc, silica, sodium silicoaluminate and clay. It is also desirable that the shaving composition is substantially free of polyethoxylated fatty alcohols. By “substantially free” is meant that the composition contains less than about 1% by weight of the named component, preferably less than about 0.5% by weight, and most preferably about 0%.

The invention may be further described by the following examples in which all parts and percentages are by weight

Examples 1-4-Aerosol Shave Foam

| | Weight Percent | | | |
|---|---|---|---|---|
| Ingredient | Ex. 1 | Ex. 2 | Ex. 3 | Ex. 4 |
| Polyglyceryl-10 Oleate | 3.90 | 2.93 | 1.95 | 2.93 |
| Behenyl alcohol | 2.93 | | | 1.46 |
| Stearyl alcohol | 2.93 | | | 1.46 |
| Cetearyl alcohol | | 3.90 | 2.93 | |
| PPG-2 Hydroxyethyl | | 1.46 | | |
| Coco/Isostearamide | | | | |
| PPG-3 Hydroxyethyl | | | 1.46 | 1.46 |
| Linoleamide | | | | |
| Sorbitan stearate | 0.98 | | | |
| Sucrose stearate | | 0.98 | 0.98 | |
| PEG-150 distearate | | 0.49 | 0.49 | |
| Pentaerythrityl | | | | 0.49 |
| tetraisostearate | | | | |
| Fragrance | | 0.39 | 0.29 | 0.39 |
| Preservative/Antioxidant | | 0.09 | | 0.09 |
| Propellant A-46 | 2.50 | 2.50 | 2.50 | 2.50 |
| Water | 87.74 | 87.26 | 89.40 | 88.24 |

An oil-in-water emulsion is formed by adding the oil phase components, which have been premelted at about 75° C., to the aqueous phase components at about 75-80° C. and mixing for about 30 minutes. The emulsion is then cooled to about 35-40° C., fragrance added, then the emulsion is homogenized to obtain a microvesicular structure (microvesicles <500 nm diameter). The creamy concentrate is filled into aerosol cans, which are then vacuum crimped, and propellant is added through the valve.

What is claimed is:

1. A shaving composition comprising, in percent by weight, about 65% to about 94% water, about 1% to about 10% of a C₁₂₋₂₂ fatty alcohol, about 1% to about 6% of a volatile hydrocarbon propellant, and about 1% to about 10% of a polyglyceryl fatty ester surfactant.

2. The shaving composition of claim 1 wherein the polyglyceryl fatty ester surfactant comprises a polyglyceryl C₁₂₋₂₂ fatty acid ester or a polyglyceryl N-fatty acyl aminoacid ester.

3. The shaving composition of claim 1 wherein the polyglyceryl fatty ester surfactant has the formula 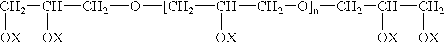
wherein n is 1 to 10, and X is a hydrogen atom or a long chain acyl group derived from a C₁₂₋₂₂ fatty acid or an N-fatty acyl-neutral amino acid, provided that at least one X is a long chain acyl group and no more than three X's are long chain acyl groups.

4. The shaving composition of claim 3 wherein, with respect to the polyglyceryl fatty ester surfactant, the long chain acyl group is derived from a C₁₂₋₂₂ fatty acid.

5. The shaving composition of claim 3 wherein, with respect to the polyglyceryl fatty ester surfactant, the long chain acyl group is selected from lauroyl, myristoyl, palmitoyl, stearoyl, isostearoyl, oleoyl, linoleoyl, and behenoyl.

6. The shaving composition of claim 3 wherein, with respect to the polyglyceryl fatty ester surfactant, the long chain acyl group is derived from an N-fatty acyl-neutral amino acid wherein the neutral amino acid is selected from glycine, alanine, β-alanine, aminobutyric acid, α-aminobutyric acid, N-methyl-β-alanine, and sarcosine.

7. The shaving composition of claim 6 wherein, with respect to the polyglyceryl fatty ester surfactant, the long chain acyl group is N-fatty acyl-sarcosyl.

8. The shaving composition of claim 3, 4, 5, 6 or 7 wherein, with respect to the polyglyceryl fatty ester surfactant, n is 4 to 8 and no more than two X's are long chain acyl groups.

9. The shaving composition of claim 3 wherein the polyglyceryl fatty ester surfactant has an HLB of at least 10 or greater.

10. The shaving composition of claim 1 wherein the polyglyceryl fatty ester surfactant comprises polyglyceryl-10 oleate.

11. The shaving composition of claim 3, 4 or 10 wherein the composition comprises, in percent by weight, about 75% to about 90% water, about 2% to about 6% of a C₁₂₋₂₂ fatty alcohol, about 2% to about 5% of a volatile hydrocarbon propellant, and about 2% to about 6% of a polyglyceryl fatty ester surfactant.

12. The shaving composition of claim 1, 3, 4 or 10 wherein the composition is substantially free of anionic surface active agents.

13. The shaving composition of claim 1, 3, 4 or 10 wherein the composition is substantially free of water insoluble pulverulent bodying agents.

14. The shaving composition of claim 1, 3, 4 or 10 wherein the composition is substantially free of polyethoxylated fatty alcohols.

15. The shaving composition of claim 3, 4 or 10 additionally comprising a sorbitan fatty ester, a sucrose fatty ester or a propoxylated fatty amide.

16. A shaving composition consisting essentially of, in percent by weight, about 65% to about 94% water, about 1% to about 10% of a C₁₂₋₂₂ fatty alcohol, about 1% to about 6% of a volatile hydrocarbon propellant, and about 1% to about 10% of a polyglyceryl fatty ester surfactant, wherein the polyglyceryl fatty ester surfactant comprises a polyglyceryl C₁₂₋₂₂ fatty acid ester or a polyglyceryl N-fatty acyl aminoacid ester.

17. The shaving composition of claim 16 additionally comprising a sorbitan fatty ester, a sucrose fatty ester or a propoxylated fatty amide.
